(19) United States
(12) Patent Application Publication
(10) Pub. No.: US 2026/0096726 A1
(43) Pub. Date: Apr. 9, 2026

(54) TRIPLE-BAND OPTICAL AUTO-CALIBRATION APPARATUS AND METHOD FOR ROBOTIC FUNDUS IMAGING

(71) Applicant: Chengdu MyBright Medical Technology Co., Ltd., Chengdu (CN)

(72) Inventor: Guangming NI, Chengdu (CN)

(73) Assignee: Chengdu MyBright Medical Technology Co., Ltd., Chengdu (CN)

(21) Appl. No.: 19/417,516

(22) Filed: Dec. 12, 2025

Related U.S. Application Data
(63) Continuation of application No. PCT/CN2025/101415, filed on Jun. 17, 2025.

(30) Foreign Application Priority Data
Sep. 20, 2024 (CN) 202411313774X

(51) Int. Cl. A61B 3/10; A61B 3/00; A61B 3/14

(52) U.S. Cl. CPC A61B 3/102; A61B 3/0091; A61B 3/14

(57) ABSTRACT

The invention discloses a triple-band optical auto-calibration fundus imaging apparatus and method for robotic ophthalmic OCT imaging, comprising: a central optical path including a fundus lens, short-pass dichroic mirror, intermediate optical calibration module I, long-pass dichroic mirror, intermediate optical calibration module II, and infrared camera; two lateral infrared cameras disposed at specific angles relative to the central optical path; an annular light ring surrounding the fundus lens; a visual induction display positioned below the long-pass dichroic mirror; an OCT sample arm with electric lift control; and an OCT optical path beneath the short-pass dichroic mirror. The apparatus captures facial, ocular, and gaze direction data, directing OCT beams through the pupil, eliminating the need for head fixation during robotic ophthalmic OCT imaging. Visual guidance reduces gaze-induced artifacts and automatically adjusts the OCT optical path to accommodate different visual acuities, enhancing robotic ophthalmic imaging efficiency and quality.

### CROSS-REFERENCE TO RELATED APPLICATIONS

The application claims priority to Chinese patent application No. 202411313774X, filed on Sep. 20, 2024, the entire contents of which are incorporated herein by reference.

### TECHNICAL FIELD

The present invention pertains to the field of optical imaging technologies, and more particularly relates to an OCT fundus imaging apparatus and method incorporating vision auto-calibration, ocular guidance, and auto-calibration for human eyes.

### BACKGROUND

Optical Coherence Tomography (OCT) enables high-resolution, non-invasive tomographic imaging of biological tissues and materials. Ophthalmic OCT has emerged as the gold standard technique for diagnosing and managing a wide range of ocular diseases. Benchtop OCT systems, leveraging their non-contact operation and high-resolution 3D imaging capabilities, are extensively utilized in ophthalmic diagnostics. However, the bulky design of benchtop systems and their dependency on skilled operators to achieve precise ocular alignment significantly restrict their application to specialized ophthalmology clinics and imaging suites. Moreover, conventional OCT systems impose stringent examination protocols on patients, including maintaining specific head and chin positions and stable gaze fixation. These requirements pose substantial challenges for individuals with cognitive or physical impairments, hindering routine ocular assessments and disease diagnosis in such populations.

Robotic OCT imaging has emerged as a promising solution to enhance imaging quality while reducing reliance on patient cooperation and operator expertise. By incorporating active eye-tracking and automated alignment mechanisms, robotic OCT systems eliminate the need for mechanical restraints on patients. Furthermore, replacing manual operation with robotic control mitigates motion artifacts caused by operator fatigue or physiological tremors, ensuring sustained imaging precision throughout the procedure. Such advancements hold transformative potential for ophthalmic healthcare delivery. However, robotic OCT systems confront a dual challenge in fundus imaging: the pursuit of broader scan coverage necessitates shorter working distances, while maintaining precise ocular alignment during imaging at these reduced distances remains a critical technical hurdle. Furthermore, the clinical imperative to accommodate subjects with varying refractive errors (including myopia and hyperopia) demands adaptive scanning protocols capable of automatically matching diverse visual profiles.

### SUMMARY

The objective of the present invention is to address the collaborative issue of achieving precise alignment between short-distance OCT imaging and the human eye. The invention provides a triple-band optical auto-calibration fundus imaging device, specifically described as follows:

A triple-band optical automatic calibration fundus imaging device comprises a central optical path. The central optical path comprises sequentially arranged components: a fundus lens, a short-pass dichroic mirror, an intermediate optical calibration module I, a long-pass dichroic mirror, an intermediate optical calibration module II, and an intermediate infrared camera. Two side infrared cameras are symmetrically disposed on both sides of the short-pass dichroic mirror, arranged at a specific angle relative to the central optical path and oriented toward the front of the fundus lens. An annular light strip is provided on the outer contour of the front end of the fundus lens. The planes of the short-pass dichroic mirror and the long-pass dichroic mirror are oriented at 45° relative to the optical paths of other components in the central optical path. The side infrared cameras are configured to capture facial and ocular images, providing partial relative positional information for aligning the imaging device with the eye pupil at an ideal working distance. The two side cameras must be positioned such that, when placed at their working distance, their respective fields of view entirely encompass the human facial area. The central optical path is configured to transmit complete ocular and pupillary images during alignment and to deliver eye-inducing light and OCT imaging beams. Within the central optical path: the short-pass dichroic mirror separates the OCT imaging beam from eye-reflected light, while the long-pass dichroic mirror separates the inducing light from eye-reflected light.

The device further comprises an inducing display positioned below the long-pass dichroic mirror, an OCT sample arm electronically controlled lifting module, and an OCT optical path located directly below the short-pass dichroic mirror. Specifically, the OCT optical path comprises components arranged from bottom to top: a collimating lens, a two-dimensional galvanometer system, and an OCT optical calibration module. The OCT sample arm lifting module drives vertical displacement of the OCT optical path. The inducing display serves to guide the eye's gaze direction. The OCT optical path controls the OCT imaging beam into a scanning state, transmits it to the short-pass dichroic mirror in the central optical path, and ultimately delivers the OCT imaging beam to the eye.

Preferably, the annular light strip is specifically implemented as multiple LED beads uniformly distributed in a circular configuration, emitting infrared light in the 750 nm-950 nm wavelength range.

Specifically, the short-pass dichroic mirror has a cut-off wavelength of 950 nm, reflecting light above 950 nm and transmitting light below 950 nm. The long-pass dichroic mirror has a cut-off wavelength of 700 nm, reflecting light below 700 nm and transmitting light above 700 nm.

Specifically, the intermediate infrared camera operates within the 750-950 nm reception band. The inducing display emits light in the 400-700 nm wavelength range. The OCT light source operates in the 1010-1110 nm spectral range. The side infrared cameras function within the 750-950 nm reception band.

Specifically, the intermediate optical calibration module I, the intermediate optical calibration module II, and the OCT optical calibration module each consist of two achromatic doublet lenses.

In another aspect, the present invention further discloses a triple-band optical self-calibration fundus imaging method, which is implemented based on the aforementioned triple-band optical auto-calibration fundus imaging apparatus. The method comprises:

The annular light strip provides infrared ambient light in the range of 750-950 nm for facial and ocular imaging with minimal interference, while projecting annular dot patterns onto the pupil to assist the central camera in pupil calibration.

Through the two lateral infrared cameras, facial images and first ocular images are acquired. Based on the facial and ocular images, the triple-band optical auto-calibration fundus imaging apparatus is controlled to reach a preliminary working position. Specifically, a robotic arm controlled by the apparatus adjusts the overall position of the apparatus.

The central infrared camera is utilized to obtain a second ocular image and the annular dot pattern via the central optical path. Based on the second ocular image and the annular dot pattern, the triple-band optical auto-calibration fundus imaging apparatus is controlled to reach the final working position. The second eye image is formed by the infrared ambient light provided by the light strip, which is reflected by the human eye and magnified through the ophthalmoscope of the central optical path. Since the direct entry into the central infrared camera would preclude the acquisition of high-quality images under an ideal field of view, the light after reflection from the eye passes through the fundus lens and then sequentially: a short-pass dichroic mirror that does not affect the transmitted light; an intermediate optical calibration module I for beam calibration; an intermediate optical calibration module II that does not affect the transmitted light; before finally being transmitted to the central infrared camera, yielding high-quality ocular images and the annular dot pattern in the pupil under an ideal field of view.

The visible-light image emitted by the visual induction display is used to induce the subject's gaze toward a specified direction. The visible light from the visual induction display is reflected into the central optical path via a long-pass dichroic mirror, then sequentially passes through: the intermediate optical calibration module I; the short-pass dichroic mirror that does not affect the transmitted light; the fundus lens; and ultimately reaches the eye to form the guidance image. The long-pass dichroic mirror is angled at 45° relative to both the central optical path and the LED visual induction display. This long-pass dichroic mirror allows the infrared band absorbed by the central infrared camera to pass through while reflecting the visible light emitted by the visual induction display.

The OCT sample arm electronically-controlled lifting module adjusts the vertical position of the entire OCT optical path based on the subject's refractive status (myopia or hyperopia) to match the optimal working distance for fundus scanning and imaging. Specifically, the OCT light source with an emission band of 1010 nm to 1110 nm emits collimated beams through a collimator. The beams are converted into parallel scanning beams in a two-dimensional plane via a two-dimensional galvanometer system, then transmitted through an OCT optical calibration module composed of two achromatic doublet lenses to the short-pass dichroic mirror in the central optical path. The short-pass dichroic mirror is angled at 45° relative to both the central optical path and the OCT lower optical. This short-pass dichroic mirror allows the infrared light received by the central infrared camera and the visible light from the visual induction display to pass through while reflecting the OCT imaging beam. The reflected OCT beam is transmitted through the fundus lens to illuminate the pupil for fundus scanning, thereby obtaining fundus tomographic images.

By adopting the aforementioned technical solution, the beneficial effects of the present invention include: The invention integrates and separates the OCT imaging optical path and the ocular calibration optical path, resolving the coordination challenge between short-distance OCT imaging and precise ocular alignment. Furthermore, the invention introduces an additional guidance optical path to direct the subject's gaze, facilitating accurate imaging of targeted regions of interest in the fundus. Additionally, based on the subject's refractive error (myopia or hyperopia), the OCT sample arm electronically-controlled lifting module adjusts the OCT optical path spacing, enabling automatic OCT fundus imaging for populations with varying visual acuity.

To make the aforementioned objectives and features of the present invention more explicit and comprehensible, the following description sets forth preferred embodiments in conjunction with the accompanying drawings to provide a detailed explanation as below.

### BRIEF DESCRIPTION OF DRAWINGS

To more clearly illustrate the technical solutions of the embodiments of the present application, the accompanying drawings used in the embodiments will be briefly described below. It should be understood that the following drawings only show certain embodiments of the present application and therefore shall not be construed as limiting the scope. For a person skilled in the art, other related drawings may be derived from these drawings without creative effort.

FIG. 1 illustrates a schematic structural diagram of a triple-band optical auto-calibration fundus imaging apparatus according to an embodiment of the present application;

FIG. 2 illustrates a schematic diagram of facial imaging using dual lateral infrared cameras according to an embodiment of the present application;

FIG. 3 illustrates a schematic effect diagram of a 16-LED annular light strip according to an embodiment of the present application;

FIG. 4 illustrates a schematic diagram of initial calibration using dual lateral infrared cameras according to an embodiment of the present application;

FIG. 5 illustrates a schematic structural diagram of an OCT imaging robot according to an embodiment of the present application;

FIG. 6 illustrates a schematic diagram of ocular imaging via the central optical path according to an embodiment of the present application;

FIG. 7 illustrates a schematic diagram of guidance light transmission to the eye according to an embodiment of the present application;

FIG. 8 illustrates a schematic diagram of precise calibration for the left eye according to an embodiment of the present application;

FIG. 9 illustrates a schematic diagram of OCT fundus imaging at a 50 mm working distance according to an embodiment of the present application;

FIG. 10 illustrates a flowchart of triple-band optical auto-calibration fundus imaging at a 50 mm working distance according to an embodiment of the present application.

Reference Numerals in the Drawings: 1. Fundus lens, 2. Short-pass dichroic mirror, 3. Intermediate optical calibration module I, 4. Long-pass dichroic mirror, 5. Intermediate optical calibration module II, 6. Central infrared camera, 7. Visual induction display, 8. Collimator, 9. Two-dimensional galvanometer system, 10. OCT optical calibration module, 11. OCT sample arm electronically-controlled lifting module, 12. Lateral infrared camera, 13. Annular light strip, 14. Eye, 15. Apparatus control robotic arm, 16. Triple-band optical auto-calibration fundus imaging apparatus.

### DETAILED DESCRIPTION OF THE EMBODIMENTS

To make the objectives, technical solutions, and advantages of the embodiments of this application clearer, the following detailed description of the technical solutions in the embodiments of this application will be provided in conjunction with the drawings in the embodiments of this application. It is evident that the described embodiments are only a part of the embodiments of this application, not all of them. The components described and illustrated in the drawings of the embodiments of this application may be arranged and designed in various different configurations. Therefore, the detailed description of the embodiments of this application provided in the following shall not be construed to limit the scope of the claims of this application but merely represent selected embodiments of this application. Based on the embodiments of this application, any other embodiments derived by those skilled in the art without requiring creative labor are within the scope of protection of this application.

First, an introduction to the applicable scenarios of this application is provided. The robotic OCT for retinal imaging of the human eye in this application is an important step in the diagnosis and treatment of eye conditions. OCT can detect subtle retinal or optic nerve head lesions, thus providing a sensitive diagnostic method in the early stages of diseases, which is crucial for early intervention and treatment of eye diseases. For diagnosed eye diseases, OCT scans can be used to monitor disease progression and evaluate treatment efficacy. For example, in glaucoma treatment, OCT can help doctors assess changes in the thickness of the retinal nerve fiber layer, thereby adjusting the treatment plan. In ophthalmic surgery, OCT can provide real-time imaging of eye structures to assist doctors with surgical planning and guidance.

Research has shown that to implement large-field robotic OCT imaging, the key is that the OCT robotic imaging device must achieve precise three-dimensional spatial positioning and tracking of the pupil and gaze direction of the eyeball within the working distance range, i.e., automatic calibration of human eye OCT imaging.

### Embodiment 1

Based on the above contents, embodiments of the present application provide a triple-band optical auto-calibration fundus imaging apparatus, which solves the recent coordination issues between OCT robotic automatic pupil calibration and OCT imaging at close distances.

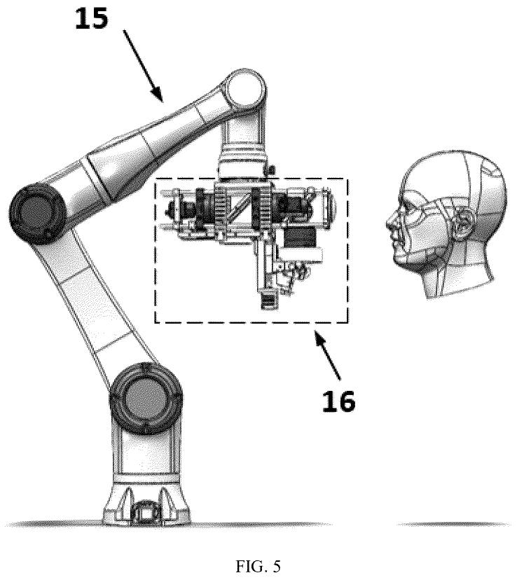

Referring to FIG. 5, FIG. 5 is a schematic structural diagram of an OCT imaging robot according to an embodiment of the present application. As shown in FIG. 5, the OCT imaging robot structure provided in this embodiment comprises: an apparatus-control robotic arm (13) and a triple-band optical auto-calibration fundus imaging device (16). The triple-band optical auto-calibration fundus imaging device is configured to acquire facial, ocular, and gaze-direction image information, as well as to implement final OCT imaging. The apparatus-control robotic arm is configured to adjust the device to an optimal working position based on the acquired facial, ocular, and gaze-direction image information.

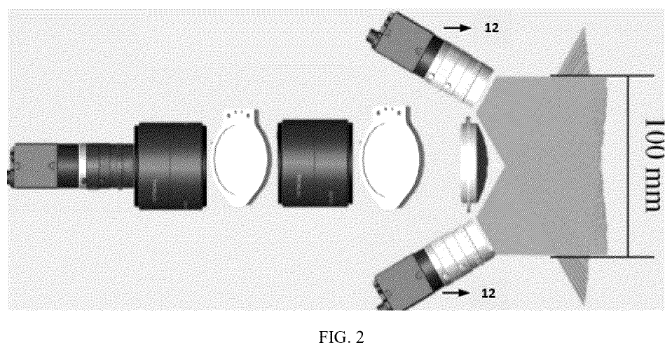

Referring to FIG. 2, FIG. 2 is a schematic diagram of facial imaging using dual lateral infrared cameras according to an embodiment. As shown in FIG. 2, the triple-band optical auto-calibration fundus imaging device includes two lateral infrared cameras (12), which achieve a 100 mm shared facial field of view at an imaging distance of 50 mm.

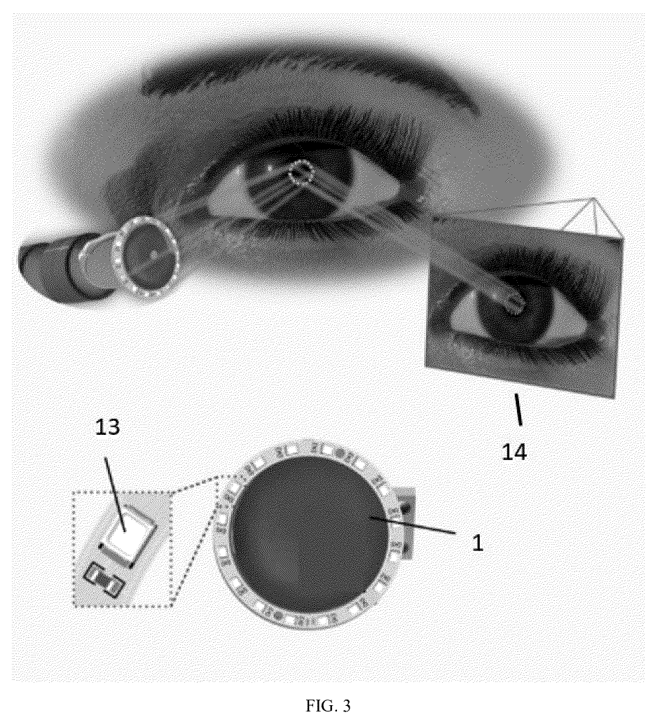

Referring to FIG. 3, FIG. 3 is the schematic diagram of an annular light strip (13) attached around the fundus lens (1). As shown in FIG. 3, the annular light strip comprises 16 evenly spaced infrared LEDs surrounding the fundus lens. In addition to providing infrared ambient illumination, it projects patterned a distinct annular dot illumination on the eye (14) to determine gaze direction for pupil calibration.

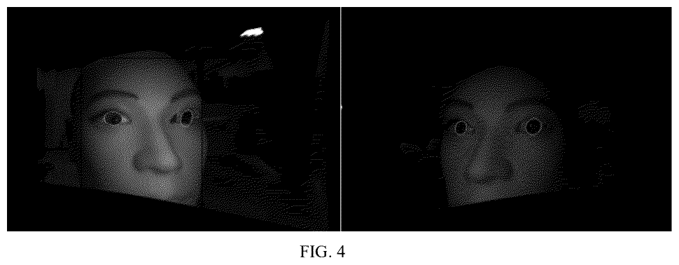

Referring to FIG. 4, FIG. 4 illustrates the initial calibration effects of the dual lateral infrared cameras. As shown in FIG. 4, the cameras acquire full facial images and complete binocular images at the initial calibration position.

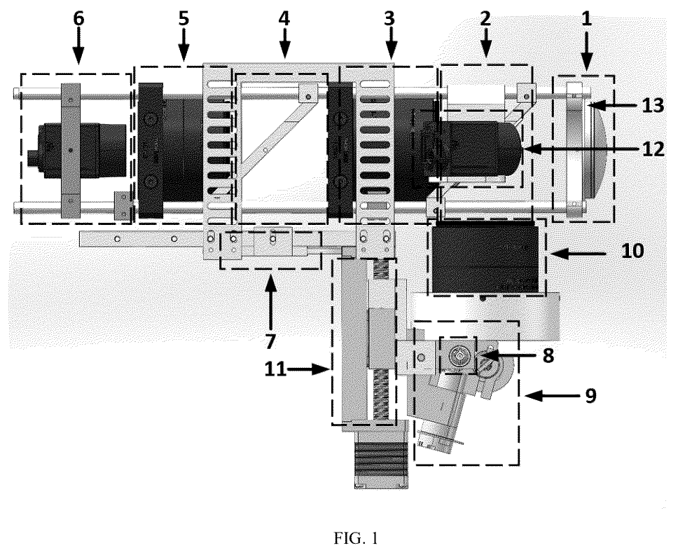

Referring to FIG. 1, FIG. 1 is a schematic structural diagram of a triple-band optical auto-calibration fundus imaging apparatus according to an embodiment of the present application. As shown in FIG. 1, the apparatus comprises a central optical path sequentially including fundus lens (1), short-pass dichroic mirror (2), intermediate optical calibration module I (3), long-pass dichroic mirror (4), intermediate optical calibration module II (5), and central infrared camera (6). Among these components, two lateral infrared cameras (12) are symmetrically arranged on both sides of the short-pass dichroic mirror (2). These cameras are oriented toward the front of the fundus lens (1) at a defined angular placement relative to the central optical path. An annular light strip (13) is disposed on the front outer contour of the fundus lens (1). The planes of the short-pass dichroic mirror (2) and long-pass dichroic mirror (4) are oriented at 45° relative to the optical axes of other components in the central optical path. Specifically, the lateral infrared cameras (12) are angled at 25-35° relative to the central optical path, with 30° adopted in this embodiment.

The apparatus further comprises: the visual induction display (7) positioned above the long-pass dichroic mirror (4), the OCT sample arm electrically controlled elevation module (11), and the OCT optical path located below the short-pass dichroic mirror (2), which comprises components sequentially arranged from bottom to top: collimator (8), two-dimensional galvanometer system (9), OCT optical calibration module (10). The OCT sample arm electrically controlled elevation module (11) is configured to drive vertical displacement of the OCT optical path.

The fundus lens (1) has a focal length of 50 mm and is configured to perform wide-field OCT retinal scanning at a 50 mm working distance.

The short-pass dichroic mirror (2), positioned centrally between the two lateral infrared cameras 12 and forming part of the central optical path, transmits induction light and 750-950 nm infrared light reflected from the eye while reflecting 1010-1110 nm OCT imaging beams.

The intermediate optical calibration module I (3) consists of two 75 mm focal length achromatic doublet lenses, serving to transmit ocular images to subsequent central optical paths and relay induction images from the visual induction display.

The long-pass dichroic mirror (4) with a cut-off wavelength of 700 nm transmits 750-950 nm infrared light reflected from the eye and reflects 400-700 nm visible light images emitted by the visual induction display (7) for guiding ocular gaze direction.

The intermediate optical calibration module II (5) directs received ocular-reflected light to the central infrared camera (6) at appropriate angles and convergence levels.

The central infrared camera (6) receives infrared ocular images reflected from the eye and transmitted through the central optical path.

The visual induction display (7) generates 400-700 nm visible light images to direct the eye's gaze toward specified orientations.

The OCT optical path is dedicated to propagating OCT imaging beams.

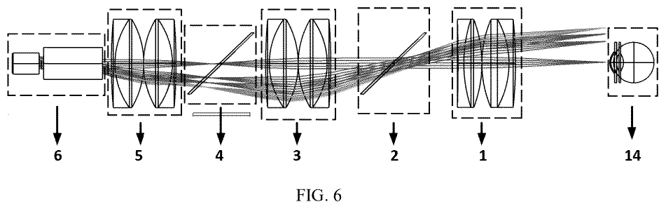

Referring to FIG. 6, FIG. 6 is a schematic diagram of a central optical path for ocular imaging according to an embodiment of the present application. As shown in FIG. 6, the eye (14) serves as the information source for OCT robotic calibration. The central optical path for ocular imaging in FIG. 6 comprises:

The fundus lens (1);

The short-pass dichroic mirror (2) with a cut-off wavelength of 950 nm, configured to transmit 400-700 nm induction light and 750-950 nm infrared light reflected from the eye while reflecting 1010-1110 nm OCT imaging beams;

The intermediate optical calibration module I (3);

The long-pass dichroic mirror (4) with a cut-off wavelength of 700 nm, configured to transmit 750-950 nm infrared light reflected from the eye while reflecting 400-700 nm visible light images emitted by the visual induction display (7) for guiding ocular gaze direction;

The intermediate optical calibration module II (5);

The central infrared camera (6).

As shown in FIG. 6, ambient light in the 750-950 nm range reflected from the eye (14) sequentially passes through the fundus lens (1), short-pass dichroic mirror (2), intermediate optical calibration module I (3), long-pass dichroic mirror (4), and intermediate optical calibration module II (5), ultimately entering the central infrared camera (6).

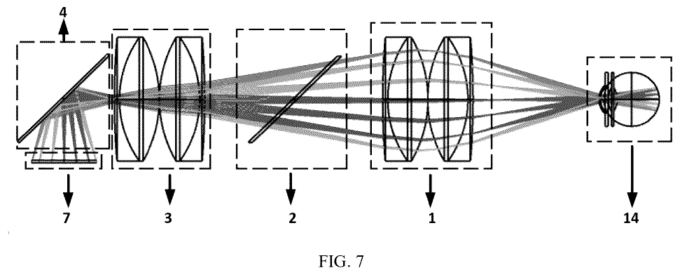

Referring to FIG. 7, FIG. 7 is a schematic diagram of induction light transmission to the eye according to an embodiment. As shown in FIG. 7, the visual induction display (7) for guiding ocular gaze emits 400-700 nm visible light images. These images first pass through the long-pass dichroic mirror (4) (700 nm cut-off wavelength), then sequentially traverse the intermediate optical calibration module I (3), short-pass dichroic mirror (2) (950 nm cut-off wavelength), and fundus lens (1), before being observed by the eye (14).

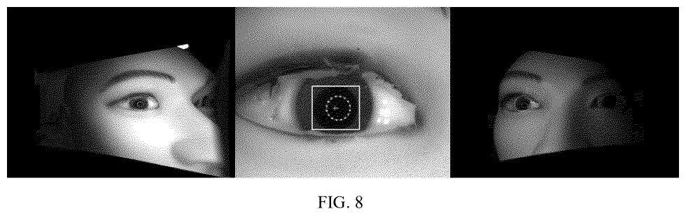

Referring to FIG. 8, FIG. 8 illustrates left-eye precision calibration effects. At the final working distance of 50 mm, the two lateral infrared cameras (12) acquire facial and ocular images, while the central infrared camera (6) captures detailed ocular imagery.

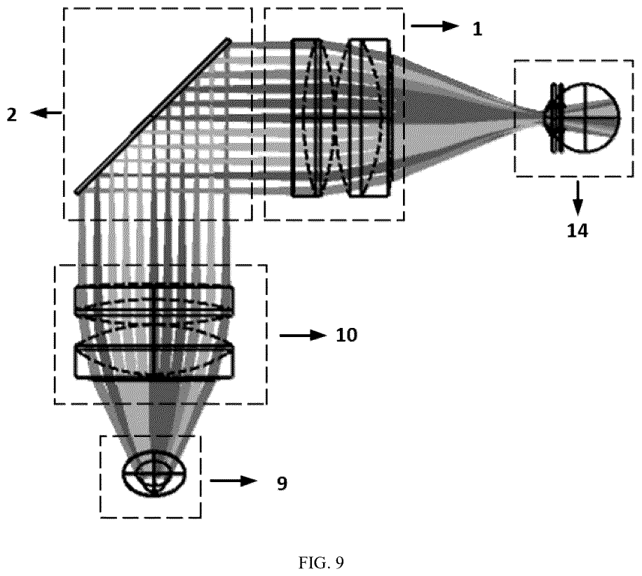

Referring to FIG. 9, FIG. 9 demonstrates OCT retinal imaging at a 50 mm working distance. The 1010-1110 nm OCT light source is collimated by the collimator (8) and incident on the 2D galvanometer system (9), transforming into a scanning state focused on the galvanometer system. The beam subsequently passes through the OCT optical calibration module (10) (comprising achromatic doublet lenses with focal lengths of 80 mm and 150 mm), reflects off the short-pass dichroic mirror (2) (950 nm cut-off wavelength) into the central optical path, traverses the fundus lens (1), and finally converges at the eye pupil position for retinal scanning at 50 mm.

### Embodiment 2

This embodiment provides a triple-band optical self-calibration fundus imaging method, implemented using the apparatus described in Embodiment 1.

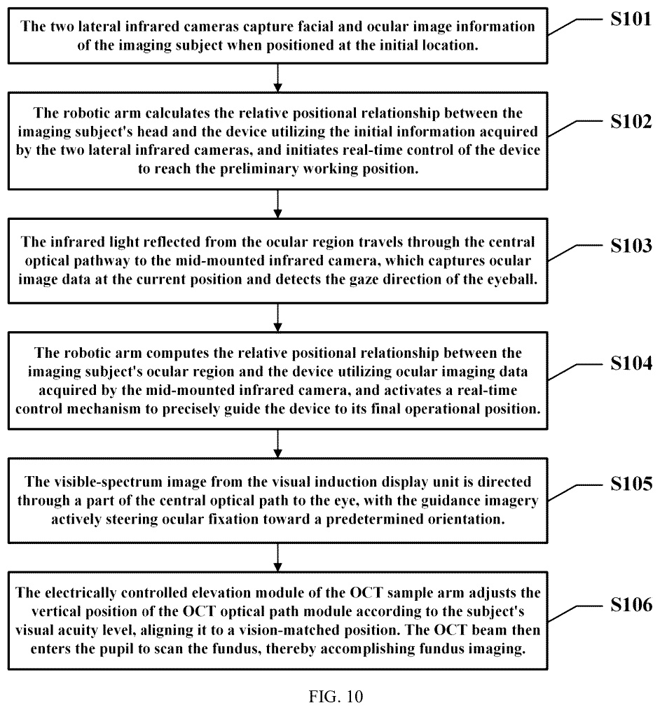

Referring to FIG. 10, FIG. 10 is a workflow diagram of triple-band optical auto-calibration fundus imaging at a 50 mm working distance. As shown in FIG. 10, the method comprises:

S101: The annular light strip (13) provides ambient illumination. The two lateral infrared cameras (12) acquire facial and ocular image information of the subject at their initial positions through imaging analogous to FIG. 2.

S102: The apparatus-control robotic arm (15) calculates the relative positional relationship between the subject's head and the apparatus using initial data from the lateral infrared cameras (12). It then initiates real-time positioning to bring the apparatus to a preliminary working position (within 10 mm of the target position). During this process, continuous feedback occurs: the lateral infrared cameras (12) update positional data, and the robotic arm (15) adjusts the apparatus accordingly.

S103: Infrared light reflected from the eye and surrounding areas travels through the central optical path to the central infrared camera (6) (as detailed in FIG. 6). The central infrared camera (6) acquires real-time ocular images and detects the annular dot pattern in the pupil for calibration.

S104: The apparatus-control robotic arm (15) further calculates the relative positional relationship between the subject's eye and the apparatus, as well as the gaze direction, using ocular images and the annular dot pattern in the pupil acquired by the central infrared camera (6). It then precisely positions the apparatus at the final 50 mm working distance (within 20 μm of the target position) through real-time control.

S105: Visible light images from the visual induction display (7) traverse portions of the central optical path to reach the eye. These images guide the subject's gaze toward predefined orientations, suppressing adverse gaze-direction fluctuations, and ultimately achieving the calibrated effect shown in FIG. 8.

S106: Based on the subject's visual acuity, the OCT sample arm electrically controlled elevation module (11) vertically adjusts the OCT optical path to an acuity-matched position. The OCT imaging beam then propagates through both the OCT optical path and a portion of the central optical path into the pupil for retinal scanning, with the beam transmission process detailed in FIG. 9.D

Positioning Requirements for the two Lateral Infrared Cameras (12): The placement and angular orientation of the two lateral infrared cameras (12) ensure full facial coverage within their fields of view at the working distance. Specifically, ≥80% of each camera's field of view must encompass facial features, with a 2-3 cm margin between the facial contour and image boundaries in this embodiment.

The above description is only a preferred embodiment of the present invention. It should be understood that the invention is not limited to the forms disclosed herein and shall not be construed as excluding other embodiments. The invention can be applied to various other combinations, modifications, and environments, and can be modified within the scope of the concepts described herein through the teachings or technical knowledge in the relevant field. Any derivative embodiments conceived by persons skilled in the art without departing from the spirit and scope of the present invention should be within the protection scope of the claims attached to the present invention.

What is claimed is:

1. A triple-band optical auto-calibration fundus imaging apparatus, characterized by comprising:
a central optical path sequentially including a fundus lens (1), a short-pass dichroic mirror (2), an intermediate optical calibration module I (3), a long-pass dichroic mirror (4), an intermediate optical calibration module II (5), and an intermediate infrared camera (6) arranged along an optical axis, wherein two lateral infrared cameras (12) symmetrically disposed on bilateral sides of the short-pass dichroic mirror (2), the lateral infrared cameras being oriented toward a front side of the fundus lens (1) and disposed at a defined placement angle relative to the central optical path; an annular light strip (13) mounted on an outer contour of a front-end portion of the fundus lens (1); wherein the short-pass dichroic mirror (2) and the long-pass dichroic mirror (4) are positioned at 45° relative to the elevation module optical paths of other components in the central optical path;
The apparatus further comprises: a visual induction display (7) positioned below the long-pass dichroic mirror (4);
The apparatus further comprises: an OCT sample arm electrically controlled elevation module (11); an OCT optical path vertically aligned beneath the short-pass dichroic mirror (2), said OCT optical path comprising a collimating lens (8), a two-dimensional galvanometer system (9), and an OCT optical calibration module (10) sequentially arranged from bottom to top, wherein the OCT sample arm electrically controlled elevation module (11) is operatively connected to the OCT optical path to drive vertical displacement thereof.

2. The apparatus according to claim 1, characterized in that the annular light strip (13) comprises a plurality of LED chips annularly and uniformly distributed.

3. The apparatus according to claim 2, characterized in that the LED chips are configured to emit infrared light in the wavelength range of 750-950 nm.

4. The apparatus according to claim 1, characterized in that: the short-pass dichroic mirror (2) has a cut-off wavelength of 950 nm, configured to reflect light above 950 nm and transmit light below 950 nm; the long-pass dichroic mirror (4) has a cut-off wavelength of 700 nm, configured to reflect light below 700 nm and transmit light above 700 nm.

5. The apparatus according to claim 1, characterized in that: the intermediate infrared camera (6) has a receiving wavelength range of 750-950 nm; the visual induction display (7) has an emission wavelength range of 400 nm to 700 nm.

6. The apparatus according to claim 1, characterized in that: the OCT light source has an emitting wavelength range of 1010 nm to 1110 nm; the lateral infrared cameras (12) have a receiving wavelength range of 750-950 nm.

7. The apparatus according to claim 2, characterized in that the intermediate optical calibration module I (3), the intermediate optical calibration module II (5), and the OCT optical calibration module (10) each comprise two achromatic doublet lenses.

8. A triple-band optical auto-calibration fundus imaging method, characterized by being implemented by the apparatus according to any one of claims 1 to 7, and comprising the steps of:
(a) providing infrared ambient light in the wavelength range of 750-950 nm through the annular light strip (13), thereby generating annular dot patterns projected from the annular light strip (13) on a pupil;
(b) acquiring facial images and first ocular images via the two lateral infrared cameras (12), controlling the apparatus to reach a preliminary working position based on said facial images and first ocular images;
(c) obtaining second ocular images and annular dot patterns through the central optical path via the intermediate infrared camera (6), controlling the apparatus to reach a final working position based on said second ocular images and annular dot patterns;
(d) guiding a subject's gaze direction toward a designated orientation using visible light images emitted by the visual induction display (7);
(e) vertically displacing the OCT optical path via the OCT sample arm electrically controlled elevation module (11) according to the refractive status of the subject, and performing OCT beam-based fundus scanning and imaging at an optimized working distance.

9. The method according to claim 8, characterized in that the lateral infrared cameras (12) are positioned and angled such that, at the working distance, their acquired images encompass the entire human face within a field of view.
